(19) United States
(12) Patent Application Publication
(10) Pub. No.: US 2013/0171066 A1
(43) Pub. Date: Jul. 4, 2013

(54) POLYMERIC COMPLEMENTS TO A B-AMYLOID PEPTIDES

(71) Applicants: Boerje Sellergren, Schwerte (DE); Carla Sofia Antunes Aureliano, Dortmund (DE); Javier Urraca Ruiz, Madrid (ES); Eric Schillinger, Dortmund (DE)

(72) Inventors: Boerje Sellergren, Schwerte (DE); Carla Sofia Antunes Aureliano, Dortmund (DE); Javier Urraca Ruiz, Madrid (ES); Eric Schillinger, Dortmund (DE)

(21) Appl. No.: 13/812,542

(22) PCT Filed: Jul. 25, 2011
(86) PCT No.: PCT/EP11/62712

§ 371 Date: Jan. 28, 2013

(30) Foreign Application Priority Data
Jul. 30, 2010 (SE) 1000791-2

(51) Int. Cl. A61K 51/06; A61K 49/12; G01N 33/68

(52) U.S. Cl. CPC A61K 51/065; G01N 33/6896; A61K 49/12
USPC 424/1.65; 530/324; 436/86; 424/9.3; 526/245; 526/292.2; 526/291; 526/293; 526/318.6; 526/306; 526/259; 525/326.2; 525/328.3; 525/329.4; 525/330.8; 525/332.2; 525/329.7

(57) ABSTRACT

The present invention relates to the preparation and use of polymer complements to β-amyloid peptides (Aβ) for detecting soluble or aggregated Aβ in fluid samples or for treating a subject having a neurodegenerative disease.

### FIELD OF TECHNOLOGY

The present invention relates to the preparation and use of polymer complements to β-amyloid peptides (Aβ) for detecting soluble or aggregated Aβ in fluid samples or for treating a subject having a neuro degenerative disease.

### BACKGROUND

Alzheimer's disease (AD) is the most common cause of late life dementia in humans and the fourth leading cause of death in the developed world. It is believed that cerebral deposition of amyloid plaques is central to the disease process. Thus, microscopically, AD is characterised by marked degeneration of the neurons and their synapses and by the presence of large numbers of senile plaques and neuro-fibrillary tangles in the cerebral neocortex and hippocampus. The plaques are made up of Amyloid deposits mainly comprising aggregates of a 39-42 residue peptide called β-amyloid (Aβ) (FIG. 1). The peptide is derived from proteolytic cleavage of the trans-membrane protein Amyloid Precursor Protein (APP), involving the two enzymes 13-secretase (at the Aβ N-terminal) and g-secretase (at the Aβ C-terminal).

The resulting peptides exhibit heterogeneous termini, with differences located at the hydrophobic C-terminus (FIG. 2). The larger, more hydrophobic, peptides (42-43 residues) are more potent for aggregation and thus plaque formation and neuronal death. Two mean values of Aβ42 in cerebrospinal fluid (CSF) in healthy or non-demented individuals have been reported, which differ by a factor of 2.5-3 (˜1650 and ˜650 pg/mL), using ELISA methodology. All studies report Aβ42 levels of AD patients to be about half the levels of the control groups although better correlations are found by measuring the ratio of soluble Aβ42/Aβ40 (K. Blennow, J. Int. Med., 2004, 256, 224). In addition to the central nervous system (CNS), amyloid peptides are found in a number of peripheral tissues. In plasma, Aβ42 is found at about ten times lower concentration than in CSF. Recent studies have shown correlations between Aβ levels in blood plasma and levels of Aβ in the brain, as well as in plaques.

An obvious therapeutic strategy is to prevent, reduce or reverse the formation of these plaques. Most drug development efforts are therefore focused on Aβ in order to either inhibit the enzymes responsible for its formation (β- and γ-secretase) to sequester the soluble fraction of the peptide or to break up existing aggregates. Although candidate drugs developed by the first and the second of these approaches are in late stage clinical trials all of the approaches taken thus far have encountered unexpected problems and doubts are raised that one single drug will cure the disease. Clearing already formed Aβ from the brain by sequestering the soluble fraction of the plaques (ADDL=amyloid derived diffusible ligands) is one of the most promising approaches. Also, removing Aβ from the blood circulation could lead to a decreased plaque burden in a patient's brain and a positive effect for the patient. Therapeutic antibodies is one approach which has shown promise in this regards. In recent years, a number monoclonal antibodies to Aβ have been developed that are capable of reducing amyloid plaque burden over time in the brains of plaque-depositing transgenic mouse models (DeMattos, R. B., et al. 2001). Peripheral anti-A beta antibody alters CNS and plasma A beta clearance and decreases brain A beta burden in a mouse model of Alzheimer's disease. Proc. Natl. Acad. Sci. U.S.A. 98:8850-8855). However, most of these antibodies target the N-terminus of Aβ leading to a general reduction of all Aβ-forms. This approach thus fails to clear specifically the most pathogenic form Aβ1-42. This indiscriminative removal of all Aβ isoforms may interfere with secondary beneficial roles that the more soluble forms of Aβ may have. Therefore treatments allowing a selective sequestration of the pathogenic Aβ1-42 form should offer therapeutic benefits. This would imply that the process of fibrillation (the aggregation of the Amyloid peptides to form plaque) could be inhibited and the disease progress hampered. The use of nanoparticles exposing a high specific surface area have proven promising in this regard (C. Cabaleiro-Lago, F. Quinlan-Pluck, I. Lynch, K. A. Dawson, S. Linse ACS Chem. Neurosci. (2010), 1, 279-287.) Thus by using amino-modified polystyrene nanoparticles of sizes ranging between 57 and 180 nm an inhibition of fibrillation was observed at higher particle concentrations. It is reasonable to assume that the onset of inhibition can be lowered by enhancing the affinity and selectivity of the nanoparticles for the Aβ42 peptide. Adequate therapy requires an accurate and early diagnosis of the disease. Thus, there is a great need for biochemical diagnostic markers (biomarkers) that could aid the clinician in the diagnosis of AD, not least at early stages in the course of the disease. Since AD pathology is restricted to the brain, cerebrospinal fluid (CSF) is an obvious source of biomarkers for AD. The CSF biomarkers total-tau protein (T-t) and different phospho-tau (P-τ) epitopes (tau being a microtubule-associated protein located in the neuronal axons) and the various forms of the peptide β-amyloid (Aβ), in particular as mentioned above the ratio of Aβ42/Aβ40 has been found to have the highest diagnostic potential (K. Blennow, J. Int. Med., 2004, 256, 224). However, practical and robust methods for accurate quantification of Aβ42 are still lacking. First the peptide is found at very low concentration in blood which demands sensitive methods. Another difficulty in the context of antibody based methods is the masking of Aβ42 epitopes by association with blood plasma proteins or by Aβ oligomerization. Hence the corresponding diagnostic methods are far from straightforward resulting commonly in insufficient selectivity, high cost, or production problems. Therefore, robust receptors capable of recognizing the epitopes under conditions promoting the dissociated form would mean significant improvements in this regard.

### SUMMARY

The objective of the present invention is to be able to remove and/or detect amyloid peptides in body fluids such as blood plasma or CSF. The objective is achieved by molecularly imprinted polymers (MIPs) selective for amyloid peptides. The invention thus discloses molecularly imprinted polymers (MIP) selective for one or more of β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (DAEFRHDSGYEVHHQKLVFFAEDVGSNKGAIIGLMVGGVVI, SEQ ID NO: 5), Aβ1-42 (SEQ ID NO: 6) or Aβ1-43 (DAEFRHDSGYEVHHQKLVFFAEDVGSN-KGAIIGLMVGGVVIAT, SEQ ID NO: 7) or a truncated isoform thereof, wherein said MIP has been prepared using a template selected from one or more of C-terminal epitope(s) or N-terminal epitope(s) of Aβ; wherein the C-terminal epitope(s) is selected from the group consisting of AβX-38, AβX-39, AβX-40, AβX-41, AβX-42 or AβX-43 or a modified form thereof; and the N-terminal epitope(s) is selected from the group consisting of (Aβ1-Y) or (Aβ2-Y) or a modified form thereof wherein X is an integer between 23 and 40 and Y is an integer between 3 and 15. In one preferred embodiment the template is selected from AβX-40 or AβX-42 or a modified form thereof.

The MIP is furthermore characterised in that it is prepared using at least one monomer, selected from a crosslinking monomer and/or a functional monomer which is polymerised in presence of the template and optionally a solvent—the template is removed from the resulting polymer, to give the molecularly imprinted polymer (MIP).

In one embodiment the MIP can selectively bind the soluble fractions (ADDL=amyloid derived diffusible ligands) of one or more of the β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (SEQ ID NO: 5), Aβ1-42 (SEQ ID NO: 6) or Aβ1-43 (SEQ ID NO: 7) or a fragment of the isoforms or any truncated isoform wherein the isoforms are present in body fluids, such as blood (serum or plasma) or cerebrospinal fluid (CSF) or in body fluids treated in order to cause protein denaturation.

The invention also discloses the use of the MIP in diagnostics and therapy of AD. The MIPs recognize preferably the N-terminal (Aβ1-X) sequence or the C-terminal (AβX-38, AβX-39, AβX-40, AβX-41, AβX-42 or AβX-43) sequence of the soluble fractions or partially aggregated fractions of Aβ or fragments of Aβ, either in body fluids like blood (serum or plasma) or CSF or in body fluids treated in order to cause protein denaturation. The MIPs can be prepared in different formats such as porous particles, nanofibers, nanotubes, monoliths, nanoparticles and their nanostructured counterparts such as core shell nanoparticles with optionally different functional properties such as magnetic or luminescent cores or labeled with probes allowing facile detection (e.g. electrochemiluminescent probes ECL). They can be tuned to exhibit maximum compatibility with the surrounding medium e.g. coatings to minimize protein adsorption.

The MIPs can be used for AD diagnostics in different ways. They can be used as selective capture phase for solid phase extraction (SPE) of plasma samples after protein denaturation. This can be followed by a protein proteolytic digestion step and detection of the peptide fragments by mass spectrometry. Alternatively the MIPs may be used in assays such as sandwich type assays where one MIP, recognizing preferentially the C-terminal epitope of Aβ, acts as a capture phase to bind Aβ, preferentially the Aβ42 isoform, and another MIP recognizing another epitope of Aβ acts as a detection phase incorporating a probe allowing a highly sensitive detection. Alternatively one of the MIPs can be replaced by an antibody recognizing the same or similar epitope thus for instance a MIP recognizing the C-terminal epitope of Aβ1-42 can be used as a capture phase and an antibody recognizing the N-terminal sequence of Aβ42 can be used for detection. Alternatively in an analogous fashion the capture phase may target the N-terminal epitope of Aβ and the detection phase the C-terminal epitope.

The MIPs can also be integrated as recognition layer in chemical sensors by combining the recognition with e.g. optical, electrical or gravimetric signal transduction.

The MIPs can also be used for therapeutic treatment of AD or another disease or disorder characterized by Aβ deposition. Examples of such disorders include: mild cognitive impairment (MCI), cerebral amyloid angiopathy or congiophylic angiopathy, Down-Syndrome associated Alzheimer's disease and inclusion-body myositis. Exemplary amyloidogenic diseases include, but are not limited to systemic amyloidosis, Alzheimer's disease, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively).

MIP based nanoparticles can be used in order to inhibit fibrillation (aggregation) of Aβ peptides or inhibiting or suppressing the neurotoxicity of Aβ or a fragment thereof by sequestering soluble Aβ peptides. The sequestering can take place from CSF or blood by direct administration of the nanoparticles or by the use of apheresis treatment of CSF or blood (extracorporal or implantable) using MIPs in a biocompatible column format (e.g. as monoliths or porous particles).

### BRIEF DESCRIPTION OF THE DRAWINGS

FIG. 1 shows a schematic representation of the amyloid precursor protein (APP) and the location of the Aβ peptide. The cleavage sites for β- and γ-secretase have been indicated.

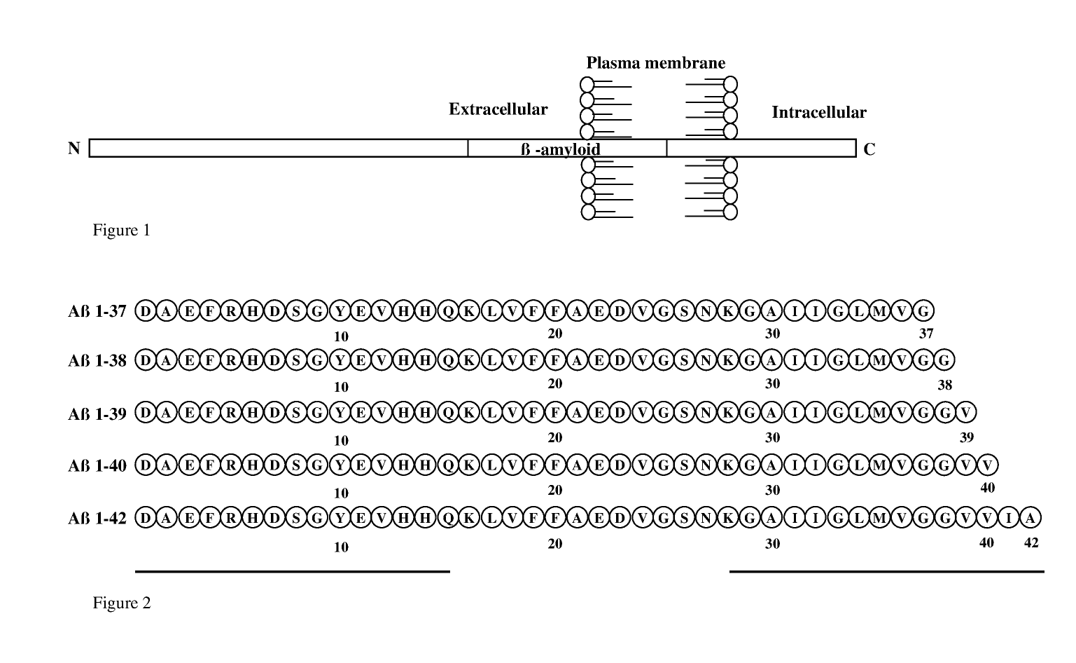

FIG. 2 shows the amino acid sequence of Aβ peptide variants resulting from the action of β- and γ-secretase on APP. The amino acid sequences corresponding to peptides that can be used as templates (modified or unmodified) for generating MIPs have been indicated. Within these amino acid sequences, shorter sequences may alternatively be chosen as templates or part of templates.

FIG. 3 shows the principle of epitope imprinting to generate free end specific binding sites for the N- or C-terminal Aβ sequences.

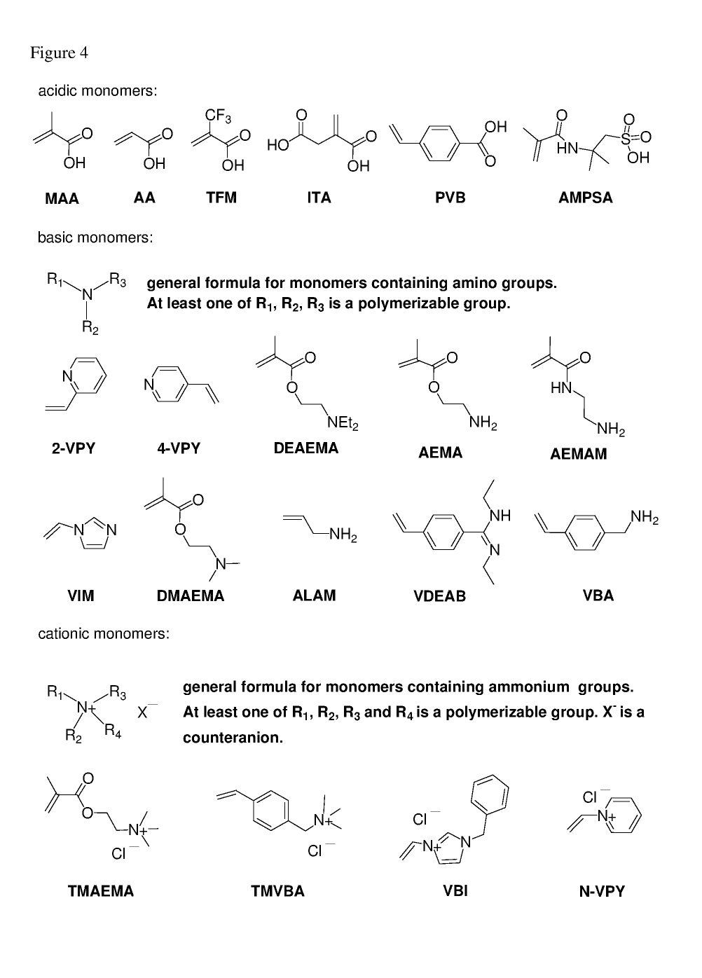

FIG. 4 shows examples of functional monomers and crosslinking monomers useful for imprinting amyloid peptides.

FIG. 5 shows the recovery versus load volume of GLMVGGVVIA (SEQ ID NO: 8) (A) and GLMVGGVV (SEQ ID NO: 9) (B) after percolation through MIP imprinted with Aβ42.

FIG. 6 shows the HPLC-UV chromatograms of elution fractions after SPE of Aβ33-40 (SEQ ID NO: 9) and Aβ33-42 (SEQ ID NO: 8) from spiked serum samples using a MIP for Aβ42 (solid line) and a corresponding NIP (dashed line).

FIG. 7 shows recoveries of Aβ1-40 (SEQ ID NO: 4) and Aβ1-42 (SEQ ID NO: 6) estimated from spot intensities from urea-SDS-PAGE/immunoblot analysis of elution fractions from SPE of a blood serum sample spiked with Aβ1-40 and Aβ1-42.

FIG. 8 shows the change in absorbance with increasing concentration of Aβ42 (closed symbols) and Aβ40 (open symbols) on a MIP for Aβ42 (squares) and a MIP for Aβ40 (circles).

### DEFINITIONS

“Body fluid” is a liquid that is inside the bodies of living people. Examples are cerebrospinal fluid (CSF), blood serum, blood plasma and urine.

“Fragments” of the Aβ isoforms refer to shorter peptides generated by for instance proteolytic cleavage of the Ab peptides.

“Denaturation” is a process in which proteins or nucleic acids lose their tertiary structure and secondary structure by application of some external stress or compound, such as a strong acid or base, a chaotropic agent, a concentrated inorganic salt, an organic solvent (e.g., alcohol, dimethylsulfoxide (DMSO) or chloroform), or heat. Examples of acids are acetic acid or formic acid, Examples of chaotropic agents are urea, guanidinium chloride and lithium perchlorate.

A “polymerisable group” is a group of atoms forming part of a monomer capable of reacting with itself or with other monomers to form a polymer.

“Specific binding” of a MIP mean that the MIP exhibits appreciable affinity for Aβ or a preferred epitope and, preferably, does not exhibit significant crossreactivity. A MIP that “does not exhibit significant crossreactivity” is one that will not appreciably bind to an undesirable entity (e.g., an undesirable proteinaceous entity). For example, a MIP that specifically binds to Aβ will appreciably bind Aβ but will not significantly react with non-Aβ proteins or peptides (e.g., non-Aβ proteins or peptides included in plaques). For example, a MIP specific for a preferred epitope will not significantly crossreact with remote epitopes on the same protein or peptide. Specific binding can be determined according to any art-recognized means for determining such binding.

The term “epitope” refers to a site on the target Aβ peptide to which, in analogy with antibodies, the MIP specifically binds. An epitope typically includes at least 3, 4, 5, 6, 7, 8, 9, 10, 11, 12, 13, 14 or 15 amino acids.

Soluble” or “dissociated” Aβ refers to non-aggregating or disaggregated Aβ polypeptide. “Insoluble” Aβ refers to aggregating Aβ polypeptide, for example, Aβ held together by noncovalent bonds. Aβ (e.g., Aβ42) is believed to aggregate, at least in part, due to the presence of hydrophobic residues at the C-terminus of the peptide (part of the transmembrane domain of APP). One method to prepare soluble Aβ is to dissolve lyophilized peptide in neat DMSO with sonication. The resulting solution is centrifuged to remove any insoluble particulates.

The abbreviation ADDL refers to amyloid derived diffusible ligands.

A “modified peptide” or a “modified form” of a peptide, for example a modified form of AβX-42, refers to a peptide modified, covalently or noncovalently, at their N- or C-terminus in order to enhance solubility or provide accessibility for the Aβ peptide when used as template.

A “support”, in the context of producing a MIP formats, refers to any inorganic support e.g. silica, alumina, porous glass, titania or an organic polymer support of any form e.g. particles, nanoparticles, nanofibers, planar supports, nanotubes.

“Nanoparticles” refers to inorganic or organic particles with an average size smaller than 1 μm (particle diameter).

### DETAILED DESCRIPTION

The invention relates to artificial receptors (molecularly imprinted polymers=MIPs) for amyloid peptides prepared via imprinting techniques and their use in diagnostics and therapy of AD or another disease or disorder characterized by Aβ deposition.

Molecular imprinting is a technique which can create robust receptors (MIPs) with antibody-like ability to bind and discriminate between molecules or other structures. This includes the copolymerisation of functional monomers with crosslinking monomers in presence of a template. The polymerisation is typically performed as free radical polymerisation where the initiating radicals are generated by a free radical initiator and optionally performed in presence of a solvent. Removal of the template from the formed polymer generates a specific cavity or binding site complementary to the template structure (e.g. size, shape and functional group) or to a target related in shape and/or functionality to the template structure.

The invention relates to molecularly imprinted polymers (MIPs) selective for one or more of β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (SEQ ID NO: 5), Aβ1-42 (SEQ ID NO: 6) or Aβ1-43 (SEQ ID NO: 7) or a truncated isoform thereof, wherein said MIP has been prepared using a template selected from one or more of C-terminal epitope(s) or N-terminal epitope(s) of Aβ; wherein the C-terminal epitope(s) is selected from the group consisting of AβX-38, AβX-39, AβX-40, AβX-41, AβX-42 or AβX-43 or a modified form thereof and the N-terminal epitope(s) is selected from the group consisting of (Aβ1-Y) or (Aβ2-Y) or a modified form thereof; wherein X is an integer between 23 and 40 and Y is an integer between 3 and 15.

In one embodiment, the template is an ammonium or quarternary ammonium salt of AβX-38, AβX-39, AβX-40, AβX-41, AβX-42, AβX-43; or (Aβ1-Y) or (Aβ2-Y) or a modified form thereof. In one embodiment, the template is selected from AβX-40 or AβX-42 or a modified form thereof. In one embodiment, the template is selected from AβX-42 (GLMVGGVVIA, SEQ ID NO: 8), AβX-40 (GLMVGGVV, SEQ ID NO: 9), Aβ37-42 (GGVVIA, SEQ ID NO: 10), Aβ35-40 (MVGGVV, SEQ ID NO: 11), and Aβ1-8 (DAEFRHDS, SEQ ID NO: 12).

In one embodiment, the MIP has been prepared using at least one monomer, selected from a crosslinking monomer and/or a functional monomer which is polymerised in presence of the template and optionally a solvent and/or the template is removed from the resulting polymer, to give the molecularly imprinted polymer (MIP). In one embodiment, the crosslinking monomer is selected from divinylbenzene. In one embodiment, the functional monomers is selected from monomers containing one or more amino groups. In one embodiment, the functional monomer is 2-aminoethylmethacrylate or a salt thereof. In one embodiment, a 1,3-disubstituted urea monomer is added as a second functional monomer. In one embodiment, the second functional monomer is N-3,5-bis(trifluoromethyl)-phenyl-N′-4-vinylphenylurea. In one embodiment, the template is immobilized to a support, such as a porous silica support, and the support is removed together with the template after polymerization. In one embodiment, the C-terminal epitope templates are bound to the support in the N-succinylated form. In one embodiment, the support is porous silica with an average pore diameter of 50 nm. In one embodiment, the MIP has been produced by grafting the polymer to the surface of a support or a nanoparticle core bead. In one embodiment, the MIP is produced in the form of nanoparticles within the size range of 10-500 nm.

The invention also relates to the use of a MIP according to any one of claims 1-14, wherein said MIP selectively binds the soluble fractions (ADDL=amyloid derived diffusible ligands) of one or more of the β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (SEQ ID NO: 5), Aβ1-42 (SEQ ID NO: 6) or Aβ1-43 (SEQ ID NO: 7) or a fragment of the isoforms or a truncated isoform wherein the isoforms are present in biological or body fluids, such as blood (serum or plasma) or cerebrospinal fluid (CSF) or in biological or body fluids treated in order to cause protein denaturation.

In one embodiment, the MIPs are used in analysis of β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (SEQ ID NO: 5), Aβ1-42 (SEQ ID NO: 6) or Aβ1-43 (SEQ ID NO: 7) or any truncated isoform or the ratio of these isoforms present in biological fluids such as blood (serum or plasma) or cerebrospinal fluid (CSF) or in biological fluids treated in order to cause protein denaturation. In one embodiment, the MIPs are used as sorbent for solid phase extraction of amyloid peptides from biological samples such as blood (plasma or serum) or cerebrospinal fluid (CSF) samples or in biological fluids treated in order to cause protein denaturation. In one embodiment, the MIPs are used as capture or detection phase in assays. In one embodiment, a MIP selective for the C-terminus of one of the β-Amyloid isoforms, such as Aβ42 or Aβ40, is used for capture and an antibody specific for the N-terminus of β-Amyloid is used for detection in a sandwhich assay. In one embodiment, the MIPs are used in chemical sensors. In one embodiment, the MIPs are used for “in vivo” imaging of amyloid deposits. In one embodiment, the MIPs are used for inhibiting the fibrillation of β-amyloid peptides.

The invention also relates to a MIP for diagnosing a disease or disorder characterised by Aβ deposition. The invention also relates to a MIP for use in treating a disease or disorder recognized by Aβ deposition. In one embodiment, the disease is Alzheimer's disease (AD).

### Composition

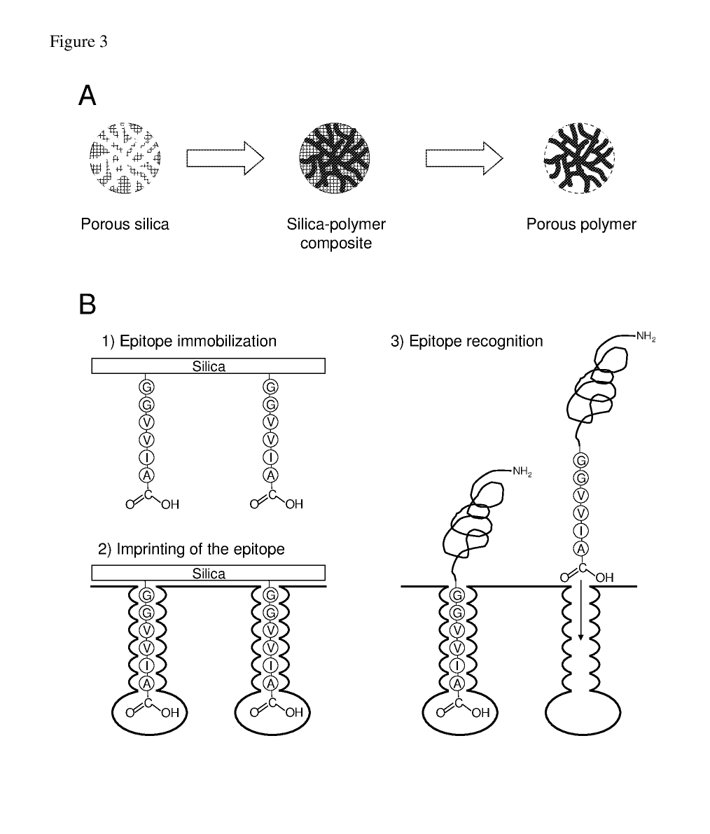

In one embodiment the template is a peptide with an amino acid sequence corresponding to Aβ or fragments of Aβ. In one embodiment the template is the Aβ N-terminal (Aβ1-Y or Aβ2-Y) sequence or the C-terminal (AβX-38, AβMX-39, AβMX-40, AβMX-41, AβMX-42 or AβMX-43) sequence (see FIG. 2) where X can be any number between 28 and 40 whereas Y preferably is an integer between 3 and 15. The peptides can be synthesised by conventional solution phase or solid phase peptide synthesis techniques and can be modified at their N- or C-terminus in order to enhance solubility or accessibility for the Aβ peptide. N-terminal modifications include any N-protecting group (e.g. Fmoc, Cbz, Boc, Ac, Bzl, Bn) whereas C-terminal modifications include conventional O-protecting group (e.g. t-butylester, methylester, ethylester, benzylester, silylgroup protection). Both the N- and/or the C-terminal modifications may also consist in the conjugation of the peptide to oligomeric or polymeric possibly dendritic bulky groups including polyethylenglycol (PEG), dextrane, polyglycerol, polyamines, polyethyleneimines or the immobilization of the peptide to the surface of support particles e.g. porous silica or silica nanoparticles. The peptide templates may also be modified by transferring them to salts. For C-terminal peptides the free carboxylic acid can be transferred to a carboxylate salt with countercations being an inorganic cation e.g. Na⁺, K⁺ or an organic cation e.g. an ammonium ion of an amine such as triethylamine, tripropylamine, tributylamine, 1,2,2,6,6-pentamethylpiperidine or a quarternary ammonium ion, e.g. tetraethyl-, tetrapropyl-, tetrabutyl-, tetrapentyl- or tetrahexyl ammonium or a quarternized aromatic heterocyclic amine such as pyridinium or imidazolium.

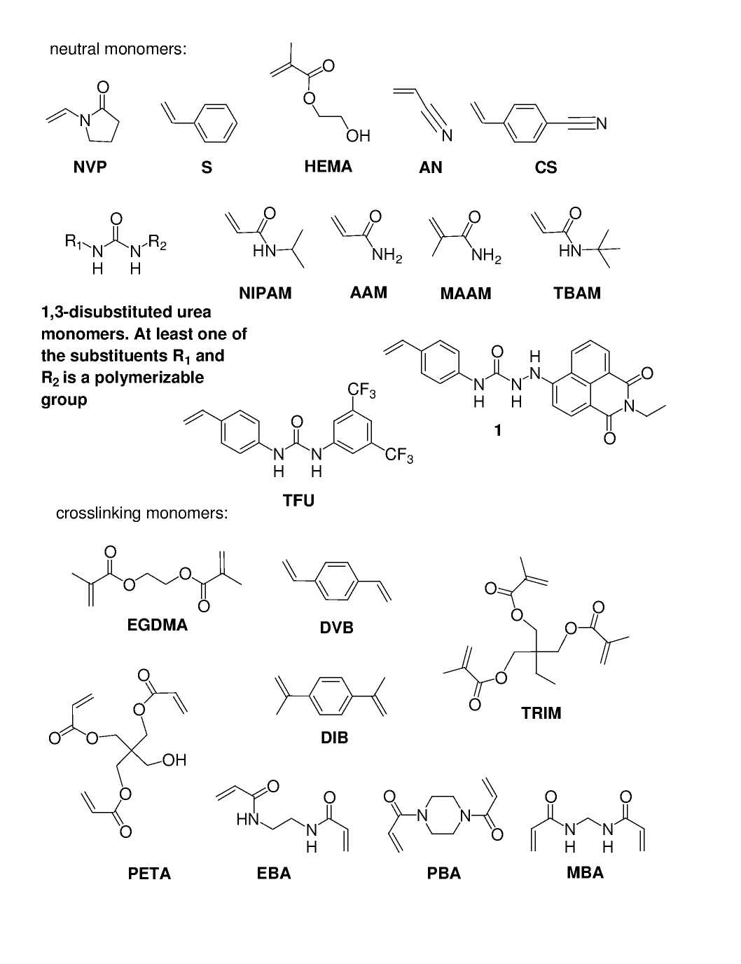

In the present invention the functional monomers are selected from monomers containing acidic functionalities (see FIG. 4) e.g. methacrylic acid (MAA), acrylic acid (AA), trifluoromethyl acrylic acid (TFM), itaconic acid (ITA), p-vinylbenzoic acid (PVB), acrylamidomethylpropane sulfonic acid (AMPSA); basic functionalities such as an amine containing monomer. Non-limiting examples are 2-vinylpyridine (2-VP), 4-vinylpyridin (4-VPY), N,N-diethyl-2-aminoethylmethacrylate (DEAEMA), 2-aminoethylmethacrylate (AEMA), N-(2-aminoethyl)methacrylamide (AEMAM), vinylimidazole (VIM), N,N-dimethyl-2-aminoethylmethacrylate (DMAEMA), allylamine (ALAM), 4-vinyl-1-(N,N′-diethylamidino)benzene (VDEAB), vinylbenzylamine (VBA); cationic monomers such as cations of salts of any amine containing functional monomer above or quarternary ammonium containing monomers e.g. N,N,N-trimethylammonium-2-ethylmethacrylate chloride (TMAEMA) N,N,N-trimethyl-N4-vinylbenzylammonium chloride (TMVBA), N-vinyl-N′-benzylimidazolium chloride (VBI), N-vinyl-pyridinium chloride (N-VPY); neutral monomers e.g. N-vinylpyrollidone (NVP), styrene (S), 2-hydroxyethylmethacrylate (HEMA), acrylonitrile (AN), cyanostyrene (CS), N-isopropylacrylamide (NIPAM), acrylamide (AAM), methacrylamide (MAAM), N-isopropylacrylamide, N-t-butylacrylamide, and monomers based on 1,3-disubstituted ureas e.g. N-3,5-bis(trifluoromethyl)-phenyl-N′-4-vinylphenylurea (TFU) or chromogenic monomer 1.

In the present invention crosslinking monomers include ethyleneglycol dimethacrylate (EGDMA), divinylbenzene (DVB), diisopropenylbenzene, (DIB), trimethylolpropanetrimethacrylate (TRIM), pentaerythritoltriacrylate (PETA), ethylenebisacrylamide (EBA), piperazinebisacrylamide (PBA), and methylenebisacrylamide (MBA).

In the present invention initiators are N,N′-disubstituted-azo-initiators e.g. azo-N,N′-bisisobutyronitrile (AIBN), azo-N,N′-bisdimethylvaleronitrile (ABDV), 2,2′-azobis-(2,4-dimethyl-4-methoxyvaleronitrile) (V70) or any other azo-based initiator (available from Wako Pure Chemical Ind. Ltd., (Japan); or redoxinitiators e.g. ammoniumpersulfate with or without added accelerator (e.g. TEMED=tetramethylethylenediamine).

Examples of solvents are water, methanol, ethanol, tetrahydrofuran (THF), acetone, dimethylformamide (DMF), acetonitrile (ACN), 1,1,1-trichloroethane, chloroform (CHCl3), dichloromethane (DCM), toluene, dimethylsulfoxide (DMSO), isopropanol or any mixture of these or other solvents.

### Formats

The MIPs can be prepared in different formats such as porous particles, nanofibers, nanotubes, monoliths, nanoparticles and their nanostructured counterparts such as core shell nanoparticles with optionally different functional properties such as magnetic or luminescent cores or labeled with probes allowing facile detection (e.g. electrochemiluminescent probes ECL). They can be tuned to exhibit maximum compatibility with the surrounding medium e.g. coatings to minimize protein adsorption. In one embodiment, the MIPs can be produced by any technique available for producing polymer particles including techniques relying on polymerisations in liquid-liquid two phase systems by suspension polymerisation or emulsion polymerisation or any variant of these (e.g. miniemulsion polymerisation), or in one liquid phase by dispersion polymerisation or precipitation polymerisation.

In another embodiment the polymerisation is performed by the technique of controlled radical polymerisation (CRP). CRP distinguish itself relative to conventional radical polymerisation in respect of the life time of the propagating radicals. In CRP this can be extended to hours which allows the preparation of polymers with predefined molecular weights, low polydispersity, controlled composition and functionality. This leads to MIPs displaying higher binding capacity, affinity and faster association and dissociation of peptide. Common CRP techniques are RAFT (reversible addition fragmentation chain transfer polymerisation), ATRP (atom transfer radical polymerisation) and iniferter (initiator, transfer, termination) polymerisation.

In another embodiment particles may be produced by grafting a MIP film on the surface of a preformed support. The grafting can be performed according to the “grafting to” or the “grafting from” approach (see above). The latter approach improves the production process as well as the molecular recognition and kinetic properties of the materials (see U.S. Pat. No. 6,759,488).

In another embodiment the particles are produced by the hierarchical imprinting approach. Here porous silica is used as a mold in order to control the particle size, shape and porosity of the resulting imprinted polymer. The template can either be immobilized to the walls of the mold or the template can be simply dissolved in the monomer mixture. The pores are here filled with a given monomer/template/initiator mixture, and after polymerisation the silica is etched away and imprinted polymer beads are obtained exhibiting molecular recognition properties. From a production stand point this procedure has the advantage of being simple and of giving a high yield of useful particles with predefined and unique morphology.

In another embodiment the MIPs can be produced in the form of nanoparticles in a typical size range of 10 nm-500 nm. The engineering of nano-micrometer-sized MIP particles can be done by precipitation polymerisation or miniemulsion polymerisation procedures where uniform imprinted beads can be produced prepared by high dilution polymerisation in one step. Both aqueous and non-aqueous imprinting protocols can be used.

Alternatively, nanoparticle synthesis can be performed by grafting. This starts from a nonporous nanosized core containing radical initiator or chain transfer groups. Onto the core a thin MIP films can be grafted with minimal influence of the bead size, dispersity and morphology on the polymerisation conditions. Nanoparticles can thus be engineered which can capture their target in vivo in the blood or CSF. Alternatively the MIPs can be used for the direct and fast extraction of the target Aβ peptide from plasma or whole blood samples. Paramagnetic, monosized polymer particles, are commonly used for this purpose. The technique is fast, mild and involves no centrifugation or chromatography. Paramagnetic MIPs may be prepared in the form of core/shell microgels by a two-stage “seed-and-feed” precipitation polymerisation or by grafting an imprinted shell on a magnetic core bead. Imprinted nanoparticles can also be designed to be incorporated in assay kits for detecting Aβ peptides. These can be based on similar principles as established immunoassays albeit with the capture antibody or detection antibody or both being replaced by a MIP. Apart from magnetic particles, the MIP particles can be labeled with probes for their facile detection. These probes can be any used in common immunoassays e.g. enzymes, radioactive isotopes, electrochemiluminescent probes based on Ruthenium or luminescent probes based on Europium.

The MIP particles can also be postfunctionalized for the purpose of biocompatibility. They can be tuned to exhibit maximum compatibility with the surrounding medium e.g. by coating the shell or outer surface with a hydrophilic coating to minimize protein adsorption and nonspecific binding. This can be in the form of so called “Stealth coatings” formed by grafting a shell consisting of polyethyleneglycol chains on the surface of the MIP nanoparticle.

### USE OF THE INVENTION

### Use of MIPs as Tools for Aβ Analysis and for Diagnosing AD or Related Diseases

The MIPs can be used for Aβ analysis and for diagnosis of AD, or any other disease or disorder characterized by Aβ deposition, in different ways. They can be used as selective capture phase for solid phase extraction (SPE) of plasma samples after protein denaturation. This can be followed by a protein digestion step and detection of the peptide fragments by mass spectrometry. Alternatively the MIP enriched sample may be subjected to an established immunoassay for quantification of the Aβ peptides. Alternatively the MIPs may be used in competitive or noncompetitive binding assays. Numerous types of competitive binding assays are known, for example: solid phase direct or indirect radioimmunoassay (RIA), sandwich type assays like solid phase direct or indirect enzyme immunoassay (EIA or ELISA), sandwich competition assay; solid phase direct biotin-avidin EIA; solid phase direct labeled assay, solid phase direct labeled sandwich assay; solid phase direct label RIA using 1-125 label; solid phase direct biotin-avidin EIA; and direct labeled RIA.

In sandwich type assays one MIP, recognizing preferentially the C-terminal epitope of Aβ, can act as a capture phase to bind Aβ, preferentially the Aβ42 or Aβ40 isoforms, and another MIP recognizing another epitope of Aβ, e.g. the N-terminal 1-X fragment, acts as a detection phase incorporating a probe allowing a highly sensitive detection. Alternatively one of the MIPs can be replaced by an antibody recognizing the same or similar epitope thus for instance a MIP recognizing the C-terminal epitope of Aβ1-42 is in one embodiment used as a capture phase and an antibody recognizing the N-terminal sequence of Aβ42 is used for detection. Alternatively in an analogous fashion the capture phase may target the N-terminal epitope of Aβ and the detection phase the C-terminal epitope.

The MIPs can also be integrated as recognition layer in chemical sensors by combining the recognition with e.g. optical, electrical or gravimetric signal transduction.

### Use of MIPs for Therapeutic Treatment of AD or Related Diseases

The MIPs can also be used for therapeutic treatment of AD or another disease or disorder characterized by Aβ deposition. Examples of such disorders include: mild congnitive impairment (MCI), cerebral amyloid angiopathy or congiophylic angiopathy, Down-Syndrome associated Alzheimer's disease and inclusion-body myositis. Exemplary amyloidogenic diseases include, but are not limited to systemic amyloidosis, Alzheimer's disease, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively).

MIP based nanoparticles can be used in order to inhibit fibrillation (aggregation) of Aβ peptides (C. Cabaleiro-Lago, F. Quinlan-Pluck, I. Lynch, K. A. Dawson, S. Linse ACS Chem. Neurosci. (2010), 1, 279-287) or inhibiting or suppressing the neurotoxicity of Aβ or a fragment thereof by sequestering soluble Aβ peptides. The sequestering can take place from CSF or blood by direct administration of the nanoparticles or by the use of apheresis treatment of CSF or blood (extracorporal or implantable) using MIPs in a biocompatible column format (e.g. as monoliths or porous particles).

The treatment of Alzheimer's and other amyloidogenic diseases may involve the administration of MIPs with affinity for specific epitopes within Aβ to a patient under conditions that generate a beneficial therapeutic response in a patient. The term “treatment” as used herein, is defined as the application or administration of a therapeutic agent to a patient, apheresis of CSF or blood for removal of pathogenic Aβ peptides or application or administration of a therapeutic agent to an isolated tissue or cell line from a patient with the purpose to cure, heal, alleviate, relieve, alter, remedy, ameliorate, improve or affect the disease, the symptoms of disease or the predisposition toward disease. Therapeutic agents of the invention include MIPs that specifically bind to Aβ or other component of amyloid plaques. In one embodiment the MIP binds specifically to the aggregated form of Aβ without binding to the soluble form. In one embodiment the MIP binds specifically to the soluble form without binding to the aggregated form. In one embodiment the MIP binds to both aggregated and soluble forms. In one embodiment the MIP binds to the naturally occurring short form of Aβ (i.e., Aβ39, 40 or 41) without binding to the naturally occurring long form of Aβ (i.e., Aβ42 and Aβ43). In one embodiment the MIP binds to the long form of Aβ without binding to the short form.

### In Vivo Imaging

The invention also provides a method for in vivo imaging of amyloid deposits in a patient. Such methods are useful to diagnose or confirm diagnosis of Alzheimer's disease. For example, the methods can be used on a patient presenting with symptoms of dementia. If the patient has abnormal amyloid deposits, then the patient is likely suffering from Alzheimer's disease. The methods can also be used on asymptomatic patients. Presence of abnormal deposits of amyloid indicates susceptibility to future symptomatic disease. The methods are also useful for monitoring disease progression and/or response to treatment in patients who have been previously diagnosed with Alzheimer's disease. The method works by administering a MIP towards Aβ which is labeled. The choice of label depends on the means of detection. For example, a fluorescent label is suitable for optical detection. Use of paramagnetic labels is suitable for tomographic detection without surgical intervention. Radioactive labels can also be detected using PET or SPECT.

The present invention will be more fully described by the following non-limiting examples.

### Example 1

### MIP Targeting the C-Terminus of Aβ42 or Aβ40

The MIP complementary to Aβ42 was prepared in the following manner. Ac-GGVVIA (SEQ ID NO: 10) (8 mg), N-3,5-bis(trifluoromethyl)-phenyl-N′-4-vinylphenylurea (TFU) (5 mg), tetrabutylammonium (TBA) chloride (2M) in methanol (2 μL) and the HCl salt of 2-aminoethylmethacrylate (AEMA), (235 mg), were dissolved in DMSO (600 μL) and ACN (900 μL). Divinylbenzene (DVB) (930 μL) and the free radical initiator ABDV (12.0 mg, 1% (w/w) total monomers) were then added. After dissolution, the solution was transferred to a glass tube, cooled to 0° C. and purged with nitrogen for 10 min. The glass tube was then sealed and polymerisation initiated thermally by placing the tube in a water bath set at 50° C. Polymerisation was allowed to proceed at this temperature for 48 h. The tube was then broken and the MIP monolith crushed into smaller fragments. The template molecule was removed through the following sequential washing steps: MeOH (100 mL), MeOH/water (0.1M HCl) (90/10, v/v) (100 mL) and finally MeOH (100 mL). The MIP particles were allowed to equilibrate for ca. 6 h with each washing solution, after which the wash solution was decanted off. Thereafter, the resulting bulk polymers were grounded and sieved to a final size ranging between 25 and 50 μm. Prior to use, they were sedimented using MeOH/water (80/20, v/v) in order to remove fine particles. A non-imprinted polymer was prepared in the same way, but in the absence of the template molecule.

A MIP for the Aβ40 was prepared analogously but using the N-acetylated hexapeptide AcMVGGVV (SEQ ID NO: 11) as template.

### Example 2

### MIP Targeting the C-Terminus of Aβ42 or Aβ40

The MIP complementary to Aβ42 was prepared in the following manner. Ac-GGVVIA (8 mg), tetrabutylammonium (TBA) chloride (2M) in methanol (2 μL) and the HCl salt of 2-aminoethylmethacrylate (AEMA), (235 mg), were dissolved in DMSO (600 μL) and ACN (900 μL). Divinylbenzene (DVB) (930 μL) and the free radical initiator ABDV (12.0 mg, 1% (w/w) total monomers) were then added. After dissolution, the solution was transferred to a glass tube, cooled to 0° C. and purged with nitrogen for 10 min. The glass tube was then sealed and polymerisation initiated thermally by placing the tube in a water bath set at 50° C. Polymerisation was allowed to proceed at this temperature for 48 h. The tube was then broken and the MIP monolith crushed into smaller fragments. The template molecule was removed through the following sequential washing steps: MeOH (100 mL), MeOH/water (0.1 MHCl) (90/10, v/v) (100 mL) and finally MeOH (100 mL). The MIP particles were allowed to equilibrate for ca. 6 h with each washing solution, after which the wash solution was decanted off. Thereafter, the resulting bulk polymers were grounded and sieved to a final size ranging between 25 and 50 μm. Prior to use, they were sedimented using MeOH/water (80/20, v/v) in order to remove fine particles. A non-imprinted polymer was prepared in the same way, but in the absence of the template molecule.

A MIP for the Aβ40 was prepared analogously but using the N-acetylated hexapeptide AcMVGGVV (SEQ ID NO: 11) as template.

### Example 3

### MIP Targeting the C-Terminus of Aβ42 or Aβ40

The MIPs complementary to Aβ40 and Aβ42 were prepared in the same manner as described in Example 1 or 2 but replacing the HCl salt of 2-aminoethylmethacrylate with the HCl salt of N-(2-aminoethyl)methacrylamide (AEMAM) (235 mg).

### Example 4

### MIP Targeting the C-Terminus of Aβ42 or Aβ40

The MIPs complementary to Aβ40 and Aβ42 were prepared in the same manner as described in Example 1 or 2 but replacing the HCl salt of 2-aminoethylmethacrylate with the HCl salt of vinylbenzylamine.

### Example 5

### MIP Targeting the C-Terminus of Aβ42 or Aβ40

The MIPs complementary to Aβ40 and Aβ42 were prepared in the same manner as described in Example 1 or 2 but replacing the HCl salt of 2-aminoethylmethacrylate with N,N,N-trimethyl-N-4-vinylbenzylammonium chloride.

### Example 6

### MIP Targeting the C-Terminus of Aβ42 or Aβ40

The MIPs complementary to Aβ40 and Aβ42 were prepared in the same manner as described in Example 1 or 2 but replacing the HCl salt of 2-aminoethylmethacrylate with methacrylic acid (MAA).

### Example 7

### MIP Targeting the N-Terminus of Aβ42 or Aβ40

The MIP complementary to Aβ1-8 was prepared in the following manner. Asp-Ala-Glu-Phe-Arg-His-Asp-Ser (SEQ ID NO: 12) (12 mg), methacrylic acid (2 mmol), methacrylamide (20 mmol), ethylbisacrylamide (20 mmol) and ammoniumpersulfate (APS) (1% wt of total monomers) were dissolved in 6 mL pH7 Hepes buffer (0.1M). The solutions were degassed for 20 min under nitrogen flow in an ice bath. Thereafter, TEMED was injected to the solution which was thereafter vigorously mixed. Then, the polymerisation was allowed to proceed at 37° C. for at least 16 h. After completion of the polymerisation, the polymer was dried and crushed followed by removal of the template by washing in MeOH, MeOH/water:1/1, MeOH/0.1M HCl (1/1) and MeOH/water.

### Example 8

### Signalling MIP Targeting the C-Terminus of Aβ42 or Aβ40

The MIP complementary to Aβ42 was prepared as described in Example 1 but replacing N-3,5-bis(trifluoromethyl)-phenyl-N′-4-vinylphenylurea (TFU) with chromogenic urea monomer 1 (see FIG. 4).

### Example 9

### Grafting of Thin MIP Films on Porous Silica Supports

Porous silica particles (average pore diameter (d)=50 nm) were modified with RAFT agent in two steps, before grafting of a polymer film on its surface.

Prior to the first modification step, the silica surface was rexydroxylated according to standard procedures. A maximum of half the silanol groups reacted with (3-aminopropyl)triethoxysilane (APS) in the first silanization steps. The subsequent step was the attachment of the RAFT agent. To a THF slurry (30 mL) of the amino-functionalized silica particles was added dropwise a THF solution (30 mL) of mercaptothiazoline activated 4-cyanopentanoic acid dithiobenzoate CPDB (0.50 g, 1.3 mmol) at room temperature. After complete addition, the solution was stirred for 6 h. The modified silica particles were recovered by filtration. 1 g of this RAFT-modified silica particles was placed in thick walled glass tubes and suspended in a polymerisation mixture according to Example 1-7 where the total monomer amount was adjusted to achieve films with minimum thickness of 1, 2 or 3 nm assuming quantitative conversion of monomer. After sealing, mixing and purging the mixture by three freeze thaw cycles, polymerisation was initiated by heating the tubes to 50° C. for 12 h and 70° C. for 12 h. After polymerisation, the samples were extracted with methanol using a Soxhlet apparatus for 24 h. Non-imprinted control polymer composites (NIP) were prepared as described above but without addition of the template.

### Example 10

### Hierarchical Imprinting of Aβ Epitopes

### A. Immobilizing Peptide Epitopes Corresponding to Aβ40 or Aβ42.

Using aminopropyl modified silica (APS-Si) with an average pore size of 50 nm as a common support material, and with the targeted surface coverage of the peptides being set to 0.2 mmol/m², peptides corresponding to the N- or C-terminal sequences of Aβ40 or Aβ42 were immobilized as follows:

### Aβ1-8:

To a suspension of 1.5 g of APS-Si and Fmoc-Asp(OtBu)-Ala-Glu(OtBu)-Phe-Arg(pbf)-His(Trt)-Asp(OtBu)-Ser (Trt)-OH (SEQ ID NO: 12) (20 mmol) in dry DMF under nitrogen, a solution of PyBop (20 mmol) and HOBt (20 mmol) and DIPEA (40 mmol) dissolved in dry DMF was added dropwise. The reaction was left under nitrogen overnight. The solid product was washed with DMF, DCM, and methanol and dried. Nonreacted amino groups were endcapped by acetalytion with acetic anhydride. This was followed by consecutive deprotection of the peptide using piperidince followed by TFA/DCM:1/1.

### Aβ36-42:

To a suspension of 1.5 g of APS-Si and the N-succinylated template SucGGVVIAOtBut (SEQ ID NO: 10) (20 mmol) in dry DMF under nitrogen, a solution of PyBop (20 mmol) and HOBt (20 mmol) and DIPEA (40 mmol) dissolved in dry DMF was added dropwise. The reaction was left under nitrogen overnight. The solid product was washed with DMF, DCM, and MeOH and dried. Nonreacted amino groups were endcapped by acetylation with acetic anhydride. This was followed by complete deprotection of the peptide using TFA/DCM:1/1.

### Aβ34-40:

To a suspension of 1.5 g of APS-Si and the N-succinylated template SucMVGGVVOtBut (SEQ ID NO: 11) (20 mmol) in dry DMF under nitrogen, a solution of PyBop (20 mmol) and HOBt (20 mmol) and DIPEA (40 mmol) dissolved in dry DMF was added dropwise. The reaction was left under nitrogen overnight. The solid product was washed with DMF, DCM, and MeOH and dried. Nonreacted amino groups were endcapped by acetylation with acetic anhydride. This was followed by complete deprotection of the peptide using TFA/DCM:1/1.

### B. Hierarchical Imprinting.

Subsequent to the template synthesis, the pores of the immobilized peptide templates were filled with a prepolymerisation mixture being identical to those of Examples 1-9 but leaving out the soluble template. This was followed by polymerisation at elevated temperatures.

Examples of prepolymerisation mixtures used for the different epitopes follow

### C-terminus (Aβ36-42 and Aβ34-40):

Alternative A: The HCl salt of 2-aminoethylmethacrylate (EAMA) (235 mg) and Divinylbenzene (DVB) (930 μL) dissolved in DMSO (600 μL) and ACN (900 μL). The free radical initiator was ABDV (12.0 mg, 1% (w/w) total monomers) and the polymerisation proceeded at 50° C.

Alternative B: Methacrylic acid (4 mmol) and Divinylbenzene (DVB) (20 mmol) dissolved in DMSO (1.2 mL) and ACN (1.8 mL). The free radical initiator was ABDV (1% (w/w) total monomers) and the polymerisation proceeded at 50° C.

### N-terminus (Aβ1-8):

Methacrylic acid (2 mmol), methacrylamide (20 mmol), ethylbisacrylamide (20 mmol) and ammoniumpersulfate (APS) (1% wt of total monomers) dissolved in 6 mL pH7 Hepes buffer (0.1M). The polymerisation proceeded after addition of TEMED at 30° C.

After polymerisation the silica mold was dissolved by treatment with a solution of NH₄HF₂ (aq) resulting in organic polymer beads with a size and morphology reflecting those of the original silica mold. In addition, the immobilized amino acids and peptides leave behind surface imprints leading to preferential retention of the template peptide when assessing the materials as sorbents for SPE or assays.

### Example 11

### Core Shell Nanoparticles

Starting from two silica core sizes (10-15 nm and 40-50 nm) commercially available as 30% colloidal solutions the cores were modified by an aminosilane to introduce reactive amino groups for further coupling of a carboxylic acid containing RAFT agent. Grafting was then performed under RAFT control in presence of the epitope template to generate surface imprinted core shell beads using the prepolymerisation solutions described in 1-9.

### Example 12

### SPE Method for Extracting Aβ Peptides

Solid-phase extraction cartridges (Varian, Spain), with a 1 mL volume, were packed with 20 mg of the Aβ42 complementary MIP (Example 1) or the corresponding nonimprinted polymers. The cartridges were equilibrated with 5 mL of Guanidine Hydrochloride (GuHCl 4M), and the sample from a body fluid (CSF or blood) containing the peptide, dissolved in buffer (GuHCl 4M), was percolated at a constant flow rate of 1 mL min⁻¹ with the aid of a peristaltic pump. The cartridges were washed with 0.5 mL of a mixture of buffer (GuHCl 4M)/AcN (95:5, v/v) to elute the nonspecifically retained compounds. Finally the Aβ peptides were eluted with 1 mL of a solution of 0.05 M (3% TFA) in MeOH. The cartridges were reequilibrated with 10 mL of buffer (GuHCl 4M) before a new application. The eluates from the MISPE column were directly injected into the HPLC system for analysis.

Serum was provided by Sigma-Aldrich and fortified with each peptide. Then the samples were diluted with 9 volumes of GuHCl 4M. Diluted samples were incubated 30 minutes at room temperature, prior to solid-phases extraction. Non-fortified water samples were preconcentrated and spiked with the peptide stock solutions for calibration purposes.

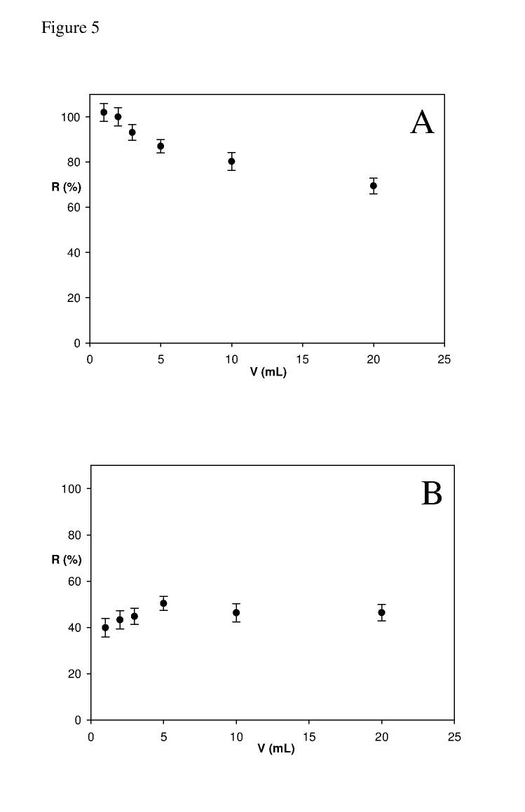

FIGS. 5 to 7 show results from the SPE method using polymers prepared as described in Example 1.

FIG. 5 shows the recovery versus load volume of 100 μg/L GLMVGGVVIA (SEQ ID NO: 8) (A) and 100 μg/L GLMVGGVV (SEQ ID NO: 9) (B) dissolved in 4M GuHCl buffer after percolation through MIP imprinted with Aβ42 (20 mg) prepared as described in Example 1.

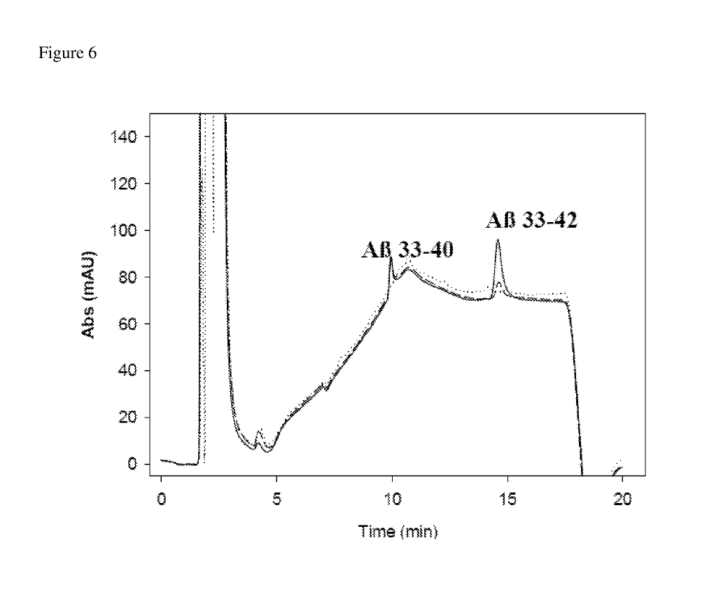

FIG. 6 shows the HPLC-UV chromatograms of elution fractions after SPE of Aβ33-40 (GLMVGGVV) (SEQ ID NO: 9) and Aβ33-42 (GLMVGGVVIA) (SEQ ID NO: 8) from spiked serum samples of 2.5 μg/mL using a MIP for Aβ42 (20 mg) prepared as described in Example 1 which is indicated by the solid line and a corresponding NIP indicated by the dashed line. The elution fraction of Aβ33-40 (GLMVGGVV) (SEQ ID NO: 9) showed a peak at ca 10 min and the elution fraction of Aβ33-42 (GLMVGGVVIA) (SEQ ID NO: 8) showed peak at ca 15 min. The dotted line represents a blank serum sample. The SPE was performed as described in Example 12.

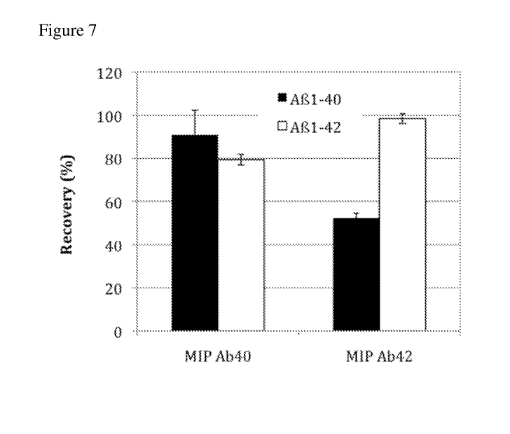

FIG. 7 shows recoveries of Aβ1-40 (SEQ ID NO: 4) and Aβ1-42 (SEQ ID NO: 6) estimated from spot intensities from urea-SDS-PAGE/immunoblot analysis of elution fractions from SPE of a blood serum sample spiked with 5 ng/mL Aβ1-40 (SEQ ID NO: 4) and 1 ng/mL Aβ1-42 (SEQ ID NO: 6). The SPE was performed as described in Example 12.

Further, for a MIP for Aβ42 prepared as described in Example 9 by grafting of a monomer mixture according to Example 1 and a corresponding NIP the following recoveries were obtained after SPE from a 4M GuHCl buffer spiked with 1 mg/L Aβ33-40 (GLMVGGVV) (SEQ ID NO: 9) and 1 mg/L Aβ33-42 (GLMVGGVVIA) (SEQ ID NO: 8) and using a washing step containing 15% acetonitrile. MIP: Aβ33-42: 100%; Aβ33-40: 36%; NIP: Aβ33-42: 59%; Aβ33-40: 34%.

### Example 13

### A Sandwich Assay Using MIP as Capture Phase and an Enzyme Linked Antibody for Detection

A MIP imprinted with Aβ37-42 (SEQ ID NO: 10) or Aβ35-40 (SEQ ID NO: 11) prepared as described in Example 10, Alternative B and a corresponding NIP was used to demonstrate the principle of MIP/antibody sandwich assays.

4 mg of polymer were placed in wells of a 96 well filter plate. 50 μl of the standard solutions containing 30-400 pg/mL Aβ1-42 (SEQ ID NO: 6) or Aβ1-40 (SEQ ID NO: 4) dissolved in 4M GuHCl were added to each well. The solutions were left in contact with the polymer in an orbital shaker during 3 hours. Then the polymers were washed with 5×200 μl of GuHCl (4M)/AcN 95:5 and 5×200 μL of milliQ water. After washing, 50 μl of an antibody conjugate solution (biotinylated antibody specific for the N-terminus of β-amyloid) were added into all wells. The plate was covered with a plate sealer and the contents of the wells mixed for a period of 5 minutes using an orbital shaker. Then the plate was incubated without shaking overnight (16-20 hours) at 2-8° C. During day 2, the sealer was removed and all the supernatant removed by filtration. 5×300 μL of a washing solution was added followed by addition of 100 μl of enzyme conjugate solution to each well. The plate was covered again with a plate sealer and incubated for 30 minutes at room temperature (20-28° C.) on an orbital shaker. Then the sealer was removed, the solutions filtered and the plate washed with 5×300 μl of a washing solution.

Finally 100 μl of substrate solution was added to each well. The plate was covered and shaken for 30 minutes. A color developed with intensity proportional to increasing concentrations of Aβ1-42. After 30 minutes 100 μL stop solution was added and the plate was agitated by hand to ensure complete mixing of the solution in all wells. After acidification, the samples were diluted with 300 μL water and filtered. Absorbance was read at 450 nm and 590 nm within 5 minutes using a plate reader ensuring absence of air bubbles.

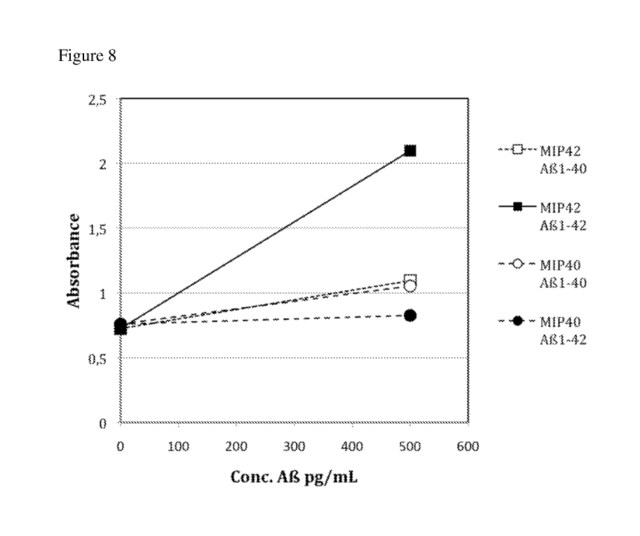

FIG. 8 shows the change in absorbance with increasing concentration of Aβ42 and Aβ40 on a MIP for Aβ42 and a MIP for Aβ40 both prepared as described in example 1.

### Analysis of CSF Samples:

CSF samples were diluted 30 times with 4M GuHCl. Diluted samples were incubated 30 minutes at room temperature, prior to ELMISA method. Also CSF samples were spiked with the peptide stock solutions for calibration purposes (30-400 pg/mL), following the standard additions method.

### Example 14

### Inhibition of Fibrillation

Fibrillation kinetics of amyloid Aβ1-42 (SEQ ID NO: 6) or Aβ1-40 (SEQ ID NO: 4) at 37° C. were monitored by the temporal development of thioflavin T (ThT) binding in the absence and in the presence of Aβ37-42 (SEQ ID NO: 10) or Aβ35-40 (SEQ ID NO: 11) imprinted polymeric nano particles prepared according to Example 11 by grafting of monomer mixtures given in Example 1. Each sample contained 10 μM Aβ1-42 (SEQ ID NO: 6) or Aβ1-40 (SEQ ID NO: 4), 200 μM ThT, and 5 μg/mL particles in 10 mM sodium phosphate buffer, 0.02% NaN₃, pH 7.4. It was observed that the presence of the particles imprinted with Aβ1-42 (SEQ ID NO: 6) strongly suppressed the fibrillation of Aβ1-42 (SEQ ID NO: 6).

1. A molecularly imprinted polymer (MIP) selective for one or more of β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (SEQ ID NO: 5), Aβ1-42 (SEQ ID NO: 6) or Aβ1-43 (SEQ ID NO: 7) or a truncated isoform thereof, wherein said MIP has been prepared using a template selected from one or more of C-terminal epitope(s) or N-terminal epitope(s) of Aβ; wherein the C-terminal epitope(s) is selected from the group consisting of AβX-38, AβX-39, AβX-40, AβX-41, AβX-42 or AβX-43 or a modified form thereof; and the N-terminal epitope(s) is selected from the group consisting of (Aβ1-Y) or (Aβ2-Y) or a modified form thereof; wherein X is an integer between 23 and 40 and Y is an integer between 3 and 15.

2. A MIP according to claim 1 wherein it has been prepared using at least one monomer, selected from a crosslinking monomer and/or a functional monomer which is polymerised in presence of the template and optionally a solvent—the template is removed from the resulting polymer, to give the molecularly imprinted polymer (MIP).

3. A MIP according to claim 1, wherein the template is an ammonium or quarternary ammonium salt of AβX-38, AβX-39, AβX-40, AβX-41, AβX-42, AβX-43; or (Aβ1-Y) or (Aβ2-Y) or a modified form thereof.

4. A MIP according to claim 1, wherein the template is selected from AβX-40 or AβX-42 or a modified form thereof.

5. A MIP according to claim 1 wherein the crosslinking monomer is selected from divinylbenzene.

6. A MIP according to claim 1 wherein the functional monomers is selected from monomers containing one or more amino groups.

7. A MIP according to claim 1 wherein the functional monomer is 2-aminoethylmethacrylate or a salt thereof.

8. A MIP according to claim 1 wherein a 1,3-disubstituted urea monomer is added as a second functional monomer.

9. A MIP according to claim 1 wherein the second functional monomer is N-3,5-bis(trifluoromethyl)-phenyl-N′-4-vinylphenylurea.

10. A MIP according to claim 1 wherein the template is immobilized to a support.

11. A MIP according to claim 1 wherein the MIP has been produced by grafting the polymer to the surface of a support or a nanoparticle core bead.

12. Use of a MIP according to claim 1 wherein said MIP selectively binds the soluble fractions (ADDL=amyloid derived diffusible ligands) of one or more of the β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (SEQ ID NO: 5), Aβ1-42 (SEQ ID NO: 6) or Aβ1-43 (SEQ ID NO: 7) or a fragment of the isoforms or a truncated isoform wherein the isoforms are present in body fluids, such as blood (serum or plasma) or cerebrospinal fluid (CSF) or in body fluids treated in order to cause protein denaturation.

13. Use of MIPs according to claim 1 in analysis of β-amyloid (Aβ) peptide isoforms Aβ1-38 (SEQ ID NO: 2), Aβ1-39 (SEQ ID NO: 3), Aβ1-40 (SEQ ID NO: 4), Aβ1-41 (SEQ ID NO: 5), Aβ1-42 (SEQ ID NO: 6) or Aβ1-43 (SEQ ID NO: 7) or any truncated isoform or the ratio of these isoforms present in body fluids such as blood (serum or plasma) or cerebrospinal fluid (CSF) or in body fluids treated in order to cause protein denaturation.

14. Use of MIPs according to claim 1 as sorbent for solid phase extraction of amyloid peptides from blood (plasma or serum) or cerebrospinal fluid (CSF) samples.

15. Use of MIPs according to claim 1 as capture or detection phase in assays.

16. Use of MIPs according to claim 1 in chemical sensors.

17. Use of MIPs according to claim 1 for “in vivo” imaging of amyloid deposits.

18. A MIP according to claim 1 for diagnosing a disease or disorder characterised by Aβ deposition.

19. A MIP according to claim 1 for treating a disease or disorder recognized by Aβ deposition.

20. A MIP according to, claim 18 wherein said disease is Alzheimer's disease (AD).

21. A MIP according to, claim 19 wherein said disease is Alzheimer's disease (AD).


SEQUENCE LISTING (12 sequences)

SEQ ID NO: 1 - protein, 37 aa, human
DAEFRHDSGYEVHHQKLVFFAEDVGSNKGAIIGLMVG

SEQ ID NO: 2 - protein, 38 aa, human
DAEFRHDSGYEVHHQKLVFFAEDVGSNKGAIIGLMVGG

SEQ ID NO: 3 - protein, 39 aa, human
DAEFRHDSGYEVHHQKLVFFAEDVGSNKGAIIGLMVGGV

SEQ ID NO: 4 - protein, 40 aa, human
DAEFRHDSGYEVHHQKLVFFAEDVGSNKGAIIGLMVGGVV

SEQ ID NO: 5 - protein, 41 aa, human
DAEFRHDSGYEVHHQKLVFFAEDVGSNKGAIIGLMVGGVVI

SEQ ID NO: 6 - protein, 42 aa, human [sequence omitted]

SEQ ID NO: 7 - protein, 43 aa, human
DAEFRHDSGYEVHHQKLVFFAEDVGSNKGAIIGLMVGGVVIAT

SEQ ID NO: 8 - protein, 10 aa, artificial
  AB33-42, a fragment isoform or a truncated isoform of beta-amyloid, present in biological or body fluids, such as blood (serum or plasma) or cerebrospinal fluid (CSF) or in biological or body fluids treated in order to cause protein denaturation
GLMVGGVVIA

SEQ ID NO: 9 - protein, 8 aa, artificial
  AB33-40, a fragment isoform or a truncated insoform of beta-amyloid, present in biological or body fluids, such as blood (serum or plasma) or cerebrospinal fluid (CSF) or in biological or body fluids treated in order to cause protein denaturation
GLMVGGVV

SEQ ID NO: 10 - protein, 6 aa, artificial
  AB37-42, a fragment isoform or a truncated isoform of beta-amyloid, present in biological or body fluids, such as blood (serum or plasma) or cerebrospinal fluid (CSF) or in biological or body fluids treated in order to cause protein denaturation
GGVVIA

SEQ ID NO: 11 - protein, 6 aa, artificial
  A35-40, a fragment isoform or a truncated isoform of beta-amyloid, present in biological or body fluids, such as blood (serum or plasma) or cerebrospinal fluid (CSF) or in biological or body fluids treated in order to cause protein denaturation
MVGGVV

SEQ ID NO: 12 - protein, 8 aa, artificial
  A1-8, a fragment isoform or a truncated isoform of beta-amyloid, present in biological or body fluids, such as blood (serum orplasma) or cerebrospinal fluid (CSF) or in biological or body fluids treated in order to cause protein denaturation
DAEFRHDS